(19) United States
(12) Patent Application Publication
(10) Pub. No.: US 2022/0117793 A1
(43) Pub. Date: Apr. 21, 2022

(54) ABSORBENT SANITARY ARTICLE FOR BODY FLUID

(71) Applicants: Samuel Jost, Tempe, AZ (US); Evan Walman, Ontario (CA)

(72) Inventors: Samuel Jost, Tempe, AZ (US); Evan Walman, Ontario (CA)

(21) Appl. No.: 17/504,011

(22) Filed: Oct. 18, 2021

Related U.S. Application Data
(60) Provisional application No. 63/092,646, filed on Oct. 16, 2020.

(51) Int. Cl. A61F 13/00; A61L 15/24; A61L 15/46

(52) U.S. Cl. CPC A61F 13/00063; A61F 13/00017; A61L 15/46; A61L 15/24; A61F 13/00991

(57) ABSTRACT

An absorbent sanitary article for absorbing a wet electrolytic or body fluid comprising, an outer material, which can comprise an absorbent low lint material, and a substrate material having a substrate surface and a reducing agent joined with the substrate material. A second substrate material can have a second substrate surface and an oxidizing agent joined with the second substrate material. Optionally at the second substrate surface, wherein the reducing agent and the oxidizing agent can be exposed to the wet electrolytic or body fluid during at least a portion of the time the absorbent sanitary article is in use.

### CROSS-REFERENCE TO RELATED APPLICATIONS

This application claims benefit from currently pending U.S. Provisional Application No. 63/092,646 titled “ABSORBENT SANITARY ARTICLE FOR BODY FLUID” and having a filing date of Oct. 16, 2020, all of which is incorporated by reference herein.

### FIELD OF THE INVENTION

The invention relates to an absorbent sanitary article for absorbing body or biological fluids.

### BACKGROUND OF THE INVENTION

A variety of absorbent sanitary articles are known. Such sanitary articles typically comprise a solid matrix consisting of pulp or other absorbent material. Once saturated with body fluid, the matrix is a substrate for a number of microorganisms. The growth of such microorganisms can give rise to sanitary and medical problems and can lead to the development of unpleasant odors.

So as to reduce the complexity and length of the Detailed Specification, and to fully establish the state of the art in certain areas of technology, Applicant(s) herein expressly incorporate(s) by reference all of the following materials identified in each numbered paragraph below. The incorporated materials are not necessarily “prior art” and Applicant(s) expressly reserve(s) the right to swear behind any of the incorporated materials.
Microulceration, ecchymoses or mucosal tearing, Fraser et al, Hum. Reprod., 1999, 14(8): 1974-1978;
Von Glahn, et al, Am. J. Pathol., 1949, 25(4): 575-595;
Dimmick et al, N. Engl. J. Med., 1975, 27; 292(13):685-687.
Qui, et al, J. Applied Polymer Science, 2007, 105(2):527-532
U.S. Pat. No. 6,488,812.
U.S. Pat. No. 7,326,314.
U.S. Pat. No. 7,794,565.
U.S. Pat. No. 7,803,249.
U.S. Pat. No. 6,264,97.
U.S. Pat. No. 4,098,728.
U.S. Pat. No. 3,961,629
U.S. Pat. No. 3,326,823.
U.S. Pat. No. 3,848,608.
U.S. Pat. No. 4,067,342.
U.S. Pat. No. 4,619,252.
U.S. Pat. No. 5,848,985.
U.S. Pat. No. 7,672,719.
U.S. Pat. No. 7,813,806.
U.S. patent publication No. 2010/0312293.
U.S. Pat. No. 5,273,596.
U.S. Pat. No. 7,713,253.
U.S. Pat. No. 6,416,799.
U.S. Pat. No. 6,264,972.
U.S. Pat. No. 4,661,101.
U.S. Pat. No. 6,710,220.
U.S. Pat. No. 6,887,564.
EP-A-348 978.
EP-A-510 619.
WO91/12029.
WO 91/11977.
WO 89/02698.
WO 91/12030.

Applicant(s) believe(s) that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(1)-(3), applicant(s) will amend the specification to expressly recite the essential material that is incorporated by reference as allowed by the applicable rules.

### BRIEF SUMMARY OF THE INVENTION

The present invention provides among other things a system for absorbing a fluid from the body of a user. It is an object of the invention to provide an absorbent bandage with improved absorbency.

It is another object of the invention to provide an antimicrobial absorbent bandage.

It is another object of the invention to provide a sanitary article with an improved absorbent bandage.

It is another object of the invention to provide a sanitary article with antimicrobial properties.

It is another object of the invention to provide an absorbent article with increased fiber surface area.

It is another object of the invention to provide a sanitary article with increased fiber surface area impregnated with antimicrobial agents.

It is another object of the invention to provide a sanitary article with needs to be changed less often.

It is another object of the invention to provide a method of manufacturing a sanitary article with improved antimicrobial and absorbent properties.

The above and other objects may be achieved using devices involving an absorbent polymer acting as a substrate for a reducing agent and an oxidizing agent. The absorbent polymer may provide a first absorbent polymer acting as a substrate for the reducing agent and a second absorbent polymer acting as a substrate for the oxidizing agent. A binder that degrades in the presence of an electrolytic solution may be used to couple the reducing agent and the oxidizing agent to the substrate.

The reducing agent and the oxidizing agent may be positioned on the substrate in a pattern and include dissimilar metals that can act as electrodes to allow the reducing agent and oxidizing agent to create a de facto battery with a voltage potential on the substrate. The reducing agent may be silver and the oxidizing agent zinc. The reducing agent may include silver particles in an amount equal to about 2% to about 6% of the substrate for the reducing agent and at least one of the reducing agent and the oxidizing agent may reside in a plurality of reservoirs on the substrate. The absorbent polymer may be selected from one or more of the group consisting of polyvinyl acetate, acrylonitrile polymer, polyacrylic acid, polyacrylamide, polyvinyl alcohol or a hydrogel. The absorbent polymer may also be grafted onto starch.

The ratio of the mass of the reducing agent to the mass of the oxidizing agent may be between about 1.7:1 to about 2.3:1 and the ratio of the reducing agent to the binder coupling the reducing agent to the substrate and the ratio of the oxidizing agent to the binder coupling the oxidizing agent to the substrate is between about 2:3 and about 7:3.

An outer material may overlay the absorbent polymer, wherein at least one of the reducing agent and the oxidizing agent is under the surface of the outer material, and the outer material may be selected from the group including an absorbent surface skin, a thin surface, and a surface layer. The outer material may be an absorbent low lint material and may comprise a nonsubstrate absorbent polymer that is not acting as a substrate for reducing agent or oxidizing agent.

The above and other object may be achieved using methods involving extruding a polymer yarn and bathing the extruded polymer yarn in a coagulation bath, spinning the polymer yarn into a textile, and adding antimicrobial elements into the textile. The polymer may be polyvinyl acetate and the antimicrobial elements may be silver and zinc. The polymer may be extruded as screw extrusion fibers and the textile may be spun into another fabric.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the. Absent such clear statements of intent to apply a “special” definition, it is the inventor's intent and desire that the simple, plain, and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for” and will also recite the word “function” (i.e., will state “means for performing the function of . . . , without also reciting in such phrases any structure, material or act in support of the function. Thus, even when the claims recite a “means for performing the function of . . . , step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 is an isometric view of the absorbent sanitary article for body fluid;

FIG. 2 is a closeup isometric view of absorbent sanitary article for body fluid;

FIG. 3 is a top view showing the anode and cathode masses of absorbent sanitary article for body fluid;

FIG. 4 is a cross section of the fibers of the absorbent sanitary article for body fluid;

FIG. 5 is a flow chart of method of manufacturing the absorbent sanitary article for body fluid;

FIG. 6 is a sample device of manufacturing a of absorbent sanitary article for body fluid; and

FIG. 7 is another sample device of manufacturing a of absorbent sanitary article for body fluid.

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

Other embodiments are as described elsewhere in the specification including the claims and other descriptions herein. As used herein and unless otherwise stated or implied by context, terms that are used herein have defined meanings or have their normally understood meanings. Unless otherwise contraindicated or implied, e.g., by including mutually exclusive elements or options, in these definitions and throughout this specification, the terms “a” and “an” mean one or more and the term “or” means and/or. As is ordinarily understood, descriptions of embodiments or articles comprising various recited elements also include those embodiments or articles consisting of or consisting essentially of those elements.

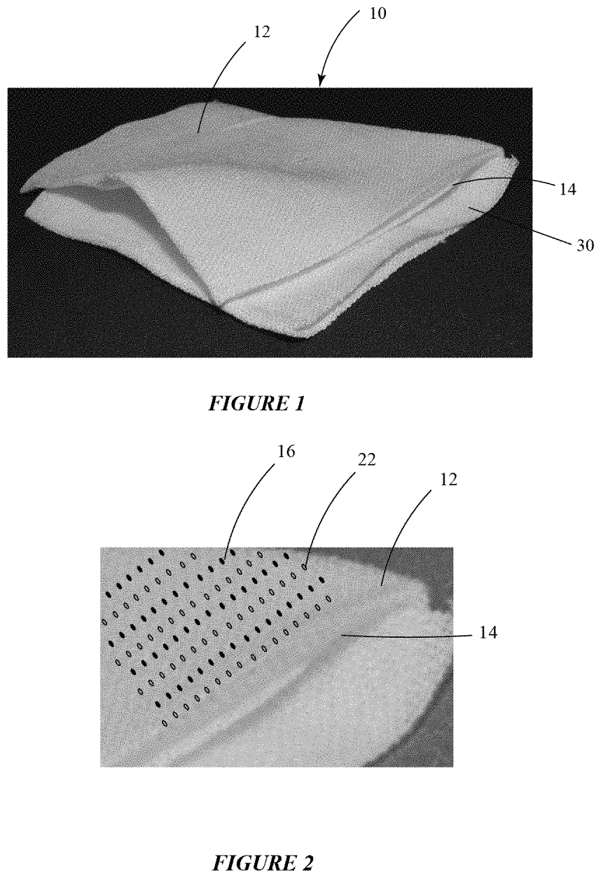

An absorbent sanitary article 10 for absorbing a fluid comprising an outer material 12 including an absorbent substrate 14 supporting a reducing agent 16 as shown in FIGS. 1-4. The absorbent substrate 14 may be composed of or blended with a low lint material. Low lint material may be cellulose acetate or vinyl. articles or cloths containing cellulose or starch or spun fibers, various synthetic polymers, e.g., polyvinyl acetate, and co-polymer materials such as starch grafted resins made from starch and acrylic acid or polyvinyl alcohol, and the like. For example, the low lint material may be a polymer yarn 30 such as polyvinyl alcohol (“PVA”) or a polyester such as polyethylene terephthalate. Fibers 18 may be created by a single or double screw extrusion followed by a coagulation bath.

The outer material 12 can comprise a woven, knitted or non-woven low lint material creating a superhydrophilic article. In certain embodiments, the substrate can be made entirely of polymer fibers, or a percentage can be put in the knitted or non-woven low lint material such as, for example, 10%, 20%, 30%, 40%, 50% or the like.

A second absorbent substrate 20 supporting an oxidizing agent 22 may also be provided. The outer material 12 can be a surface skin, a thin surface or a surface layer that covers at least a portion 13 of the absorbent sanitary article 10, which may contain other materials such as other absorbent materials, the reducing agent 16 or the oxidizing agent 22. The surface skin may a thickness of about 20-200 microns and the thin surface can have a thickness of about 0.3 to 1.5 mm. The surface layer may have a thickness between about 2-4 mm. The surface skin, thin surface or a surface layer is absorbent and may comprise channels with end pores that extend throughout its thickness.

The reducing agent 16 may be, for example, silver joined with a substrate surface, by affixing the element directly to the other element, indirectly securing the element to the other element by affixing the element to intermediate member(s) which in turn are affixed to the other element or may be one element that is integral with another element, i.e., one element is essentially part of the other element. An element directly secured to another element can be secured covalently or non-covalently, e.g., by ionic attraction and/or by entrapment of one element, e.g., silver particles, in pores or channels of the other, e.g., an absorbent polymer or fiber.

Lint is generally defined as small fibers in material to be rubbed from an article when handled or in normal use. A low lint material may be a permeable or absorbent material, that does not release lint when handled or in normal use. Low lint material may have voids extending through its surface to a depth in the material, including through the entire thickness of the low lint material. Low lint material will typically be formed of a material that is resistant to adhering to skin or mucosal tissue. Low lint material can be a calendered fabric with one or both surfaces having a mean surface roughness in the range of about 1 micron to about 200 microns.

In some embodiments of the invention the outer material 12 of the absorbent sanitary article 10 comprises low lint polyvinyl acetate absorbent material. The absorbent sanitary article 10 may also contain bio-compatible low-lint or lint-free materials, e.g., swellable materials or sponges, which may be present at least a portion the surface of the outer material 12.

Other absorbent materials that can be included in the absorbent sanitary articles include fluff pulp, cotton and linen, which can shed appreciable amounts of lint during use. These materials are preferably primarily present under the outer material 12 e.g., under the thin surface or surface layer, of the absorbent sanitary article 10. In some embodiments, the reducing agent 16 and/or the oxidizing agent 22 is dispersed within such absorbent materials. In other embodiments, the reducing agent 16 and/or the oxidizing agent 22 is dispersed in both the thin surface or surface layer of the absorbent sanitary article 10 in all or a portion of the lint shedding absorbent materials.

Absorbing materials include fluff pulp, carboxymethylcellulose, polyester and other absorbing fibers or polymers. For articles containing various absorbent polymers or hydrogels, e.g., silicone hydrogels, polyacrylamides, polyethylene oxide, poly(2-acrylamido-2-methyl-1-propanesulfonic acid, polyvinylpyrrolidone or polymerized monoethylenically unsaturated monomers bearing carboxyl groups, which may be partially neutralized. The degree of crosslinking may be used to control the article's relative absorbency and other physical properties.

The extrusion process to manufacture the polymer yarn can comprise two intermeshing, co-rotating screws mounted on splined shafts in a closed barrel. The raw material can be introduced in solid or liquid form through the feeder where contact is initiated between the new melt and the screws of the extruder. A melt pumping begins where the polymer is extruded in a viscous form followed by a coagulation bath to allow cooling. Finally, the cooled fiber can be spun where manufacturing of various applications can begin. The fibers can be manufactured by weaving, knitting or through a non-woven process or any combination thereof. The manufacturing process can comprise a weaving machine and the extrusion process, a knitting machine and the extrusion process, a non-woven machine and the extrusion process or any combination thereof.

By extruding the fibers 30 and combining them with either weaving, knitting or non-woven manufacturing process the combination of processes can create a higher surface area than other materials which can lead to higher surface tension and higher water retention within the absorbent sanitary article. The screw extrusion fibers allow for overlapping fibers to create thousands of capillaries that wick fluid away from the site of contact with the sanitary article. The fluid absorbed and retained by the article 10 upon the user's skin and mucosal contact can be distributed throughout the absorbent sanitary article to mitigate moisture buildup, reservoirs and any ultimate risk of maceration. Antimicrobial elements added into the PVA polymer material can allow fluid to be retained for greater periods of time without the risk of infection or malodor. This is in addition to the inherent bacteriostatic properties of may polymers, including PVA.

The configuration of the fibers 30 allows the fluid absorbed and retained by the article 10 upon skin and mucosal contact to be balanced and provides an optimal level of moisture content in the tissue contacting device replace with anti-flocking properties of absorbent sanitary article. The low lint material as opposed to cotton has much lower adherence to open wounds and can promote healing of the wound, which can appear from polymer being extruded as a single strand with little to no defect present in the extruding mouth. polymer materials produce less lint than other woven, knitted or other materials.

The outer material 12 may alternatively or additionally include a hydro gel. Hydro gels are cross-linked water absorbing polymers, which absorb aqueous solutions through hydrogen bonding with water molecules. A hydro gel's ability to absorb water is affected by the ionic concentration of the aqueous solution, e.g., a wet electrolyte or bodily fluid. The total absorbency of hydro gels is controlled by the type and degree of cross-linkers used to make the gel. Low density cross-linking increases the absorbent capacity and swelling. Low density cross-linked hydro gels tend to be soft and somewhat sticky. A high cross-link density exhibits lower absorbing capacity and degree of swell, but the gel strength is firmer and articles can better maintain shape under pressure. In some embodiments, absorbent hydro gels are a component of the outer material 12.

In some embodiments, the reducing agent 16 and an oxidizing agent 22 may be dissimilar metals such as, for example, silver and zinc. In the presence of a wet electrolyte or bodily fluid, the reducing agent 16 and the oxidizing agent 22 constitutes electrodes, which may be painted or printed onto a surface, e.g., a non-conductive surface. This can be accomplished to create a pattern or an array of voltaic cells that do not spontaneously react (generate an electrical current between electrodes in suitable proximity) until the electrodes are in electrical contact with each other when in the presence of the wet electrolyte or bodily fluid.

Electrodes may be painted or printed onto a non-conductive surface to create a pattern or an array of electrodes. The reducing agent 16 and the oxidizing agent 22 may be attached with a substrate surface 12 or second substrate surface 20 by printing with ink or painting with paints. Means to affix such materials are known in the printing arts, including ways to apply and cure the inks.

A binder material 40 can be attached with the oxidizing or reducing agents 16, 22. The binder material 40 may be a biocompatible liquid material that can be mixed with the oxidizing or reducing agents 16, 22, e.g., metallic crystals of silver or zinc, to create an ink which may be applied as a thin coating to a surface. One suitable binder is a solvent reducible polymer, such as the polyacrylic non-toxic silk-screen ink manufactured by Colorcon, Inc., a division of Berwind Pharmaceutical Services, Inc. (Colorcon No-Tox®, number NT28). The binder can be mixed with high purity (e.g., at least 99.999%) metallic silver crystals to make silver ink in a particular embodiment. The silver crystals are made by grinding to a powder and can be smaller than 100 microns in size. In one embodiment, the size of the crystals is about 325 mesh, which is typically about 40 microns in size. The binder 40 is separately mixed with high purity (e.g., at least 99.99%) metallic zinc powder, which has also been sifted through standard 325 mesh screen, to make the zinc ink. For better quality control, most of the crystals are preferably larger than 325 mesh and smaller than 200 mesh. In other embodiments, other metal powders can be used to make other metallic inks in essentially the same way.

The ratio of metal to binder material is used to control the release rate of the metal from the dried ink mixture. When Colorcon's polyacrylic ink is used as the binder, about 50-70% of the mixture should be metal for most envisioned uses for the absorbent sanitary articles of the invention. Other binders may dissolve or otherwise break down faster or slower than a polyacrylic ink, so adjustments can be made to achieve the desired rate of spontaneous reactions from the oxidizing and reducing agents.

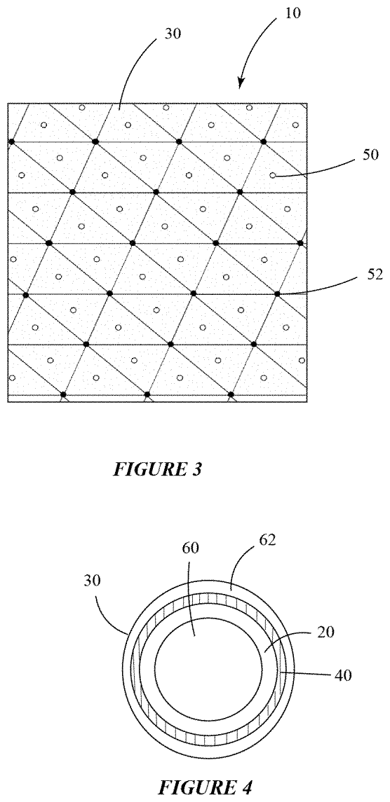

Referring to FIG. 3, when a single mass of anode metal 50 such as silver is spaced from a single mass of cathode metal 52 such as zinc, a single voltaic cell is created when a wet electrolyte electrically connects the masses. To control the number of voltaic cells a pattern of alternating anode masses and cathode masses may create an array of electrical currents across the primary surface. In one embodiment, a pattern has each mass of silver ink equally spaced from four masses of zinc ink, and has each mass of zinc ink equally spaced from four masses of silver ink. The ink can be present as round dots or polygon shapes that are repeated. In the absorbent sanitary articles the silver ink will generally have about twice as much mass of silver as the zinc ink has of zinc. When present on the same surface of a support substrate, the silver and zinc ink masses are preferably about a millimeter from each other. The resulting pattern of dissimilar metal masses defines an array of voltaic cells when introduced to an electrolytic solution. In other embodiments, the anode and cathode masses 50, 52 are not present on the same surface of a support substrate, e.g., on opposite sides of a support substrate or on different support substrates that are not attached to each other.

Other ways to affix or bind reducing or oxidizing agents to a support substrate are known to those having skill in the art. In particular embodiments, the reducing agent 16 is silver. In these embodiments silver can be chemically or physically attached to a fiber 30 surface such that it cannot be flushed away or easily removed from the fiber 30 during use of the absorbent sanitary article 10. Attachment may be via electrostatic charges. Silver-coated fiber is available for example from Statex Produktions, Vertriebs GmbH, Querlandstr. 6b, 28357 Bremen, Germany. Fiber material to which silver can be attached include polyamides such as nylon-6,6 or nylon-6, a polyester, such as Dacron, Diolen ornTrevira, a polyacrylic, such as acrylic, Dralon, Dolan or Orlon, an elastane, such as Dorlastan or Lycra, or a polychloride, such as Movil or Rhovyl. When affixed to a support substrate the reducing agent 16 can be present in relatively low amounts in the absorbent sanitary article 10.

Silver affixed to fiber material such as a polyamide or a polyester may be present as particles of about 1 nm to about 30 nm in diameter, preferably about 2 to about 10 nm. In these embodiments, smaller particle sizes for the reducing agent 16 and the oxidizing agent 22 are preferred compare to the particle sizes that are suitable for use in inks (or paints) containing the reducing agent 16 or the oxidizing agent 22.

In some embodiments, the reducing agent 16 and/or oxidizing agent 22 is fixed against movement relative to the substrate surface in the absorbent sanitary article 10. In other embodiments, the reducing agent and/or oxidizing agent is mixed with or infused into absorbent materials in the absorbent sanitary article 10.

Typically, the wet electrolytic or bodily fluid is blood, urine or feces for the absorbent sanitary articles such as sanitary pads, tampons, diapers, or the like for incontinence, absorbing menstrual fluids or other normal uses. In some embodiments, the absorbent sanitary article 10 is a sanitary napkin or tampon for absorbing menstrual fluids. In particular embodiments, the absorbent sanitary articles 10 are tampons designed to absorb predetermined amounts of blood or other menstrual fluid, e.g., less than 6 g, 6-9 g, 9-12 g, 12-15 g or 15-18 g, or other normally accepted tampon absorbency ratings.

The reducing agent 16 in the absorbent sanitary article 10 may be present in multiple reservoirs attached with the substrate material 14 and/or the oxidizing agent 22 is present in multiple second reservoirs attached with the second substrate material 20. Such reservoirs may be printed onto the surface of the substrate 14 or the surface of the second substrate material 20. The reservoirs may be in various configurations, e.g., (i) dots or other reservoirs shaped spaced about 0.3 to about 5 mm from each other along the length of the substrate material, (ii) a single continuous strip along the length of the substrate material 14 or the second substrate material 20 or (iii) small pieces of substrate material or second substrate material having, e.g., an area of about 0.5 mm2 to about 5 mm2, where all or a portion of the substrate material or second substrate material is attached with the reducing or oxidizing agent 16, 22. In other embodiments, the reducing agent 16 and the oxidizing agent 22 is present on the same surface of substrate material such as a pliable substrate, wherein the reducing agent 16 and the oxidizing agent 22 is present in a pattern of masses, e.g., alternating ink masses present as dots or adjacent polygons, alternating adjacent strips of ink, with each strip containing either the reducing agent 16 or the oxidizing agent 22.

In some embodiments, the substrate material 14 and/or second substrate material 20 is present as beads. The beads are typically composed of absorbent material, to which the reducing agent 16 or the oxidizing agent is attached 22. The reducing agent 16 or the oxidizing agent 22 can be attached at the surface of beads or disposed within, either by direct attachment to the absorbent material or by mechanical mixing such as by infusion of the reducing agent 16 or the oxidizing agent 22 into the beads as a suspension in a fluid carrier such as deionized water, glycerol or a deionized water and glycerol mixture.

The reducing agent 16 may be present on one surface of a substrate material 14 and the oxidizing agent 22 is present on the opposite surface of the substrate material 20. The substrate material can be present in various shapes such as strips of a pliable sheet, e.g., (i) about 0.5 mm to about 10 mm wide or about 1.5 mm to about 2.5 mm wide or (ii) disposed immediately under or in direct contact with the entire inner surface area of the outer material of the absorbent sanitary article 10.

The absorbent sanitary article 10 is effective in part through the presence of the reducing agent 16, either as metallic silver alone or zinc alone (when silver is not present). Silver metal is an antimicrobial agent with activity against common bacterial pathogens such as Staphylococcus aureus, group A streptococci and Pseudomonas aeruginosa. The electrochemical nature of silver is such that it is positively charged and thus is able to bind to negatively charged moieties on bacterial cells. The effect of this binding interaction between silver and bacterial proteins leads to killing of bacterial cells. When both the silver and zinc are present and the reduction-oxidation reaction is ongoing, silver metal is converted to silver ion, which is also has anti-microbial activity. When an induced electrical current is present in the absorbent sanitary article, the current electrochemically attract microbes. Bacteria and other microbes are drawn to the silver by virtue of their overall net negative charge where they die on contact with the silver.

In some embodiments, the absorbent sanitary article is a fibrous tampon comprising at least one inner layer having one or more malodor counteractant materials incorporated therein. Malodor counteractant materials include, for example, glycerin and ethylenediaminetetraacetic acid (EDTA). Other suitable malodor counteractant materials are activated carbons, clays, zeolites, silicates, cyclodextrins and ion exchange resins. The malodor counteractant materials are typically present in an amount between about 0.01 grams to about 0.12 grams, usually in a liquid form. A malodor counteractant material may also be present between about 0.01 grams to about 20% of the total weight of the tampon, e.g., about 0.01 grams to about 0.12 grams of glycerin or about 0.01 grams to about 0.05 grams of glycerin.

The low-lint or lint-free material can have a relatively uniform pore geometry and pore size distribution throughout its volume. Such material, e.g., a polyvinyl acetal or a hydrophilic polyurethane, can be prepared to have uniform swellability and/or water or fluid absorption or absorbency time ranging from a few 10 seconds to about 20 minutes. The material can optionally contain a swellable polymeric gelling agent and optionally an agent that can accelerate biodegradation, e.g., an aqueous fluid swellable polysaccharide such as dextrin, partially epoxidized starch or an inulin. Such materials can have a liquid absorption capacity greater than twenty times the material's weight. Once fluids have entered the pores of the outer material, they are retained primarily by capillary action. The presence of absorbent material under but in contact with the outer material wicks absorbed fluids into the interior of the absorbent sanitary article. When such absorbent material is shielded from bodily contact by the outer material of the absorbent sanitary article, it can be selected without regard to its tendency to shed lint or larger particles, e.g., an absorbent polymer or fiber such as fluff pulp, rayon or cotton, or mixtures thereof.

In some of the foregoing embodiments, the absorbent sanitary article is, e.g., a tampon, comprising a body made of polyvinyl acetal material with a central porous or absorbent section and the outer material of is a porous surface skin ranging from, e.g., about 20 microns to about 200 microns in thickness, preferably about 30-60 microns, surrounding at least a substantial portion or essentially all of the central section.

The substrate material 14, 20 may be encased by a stretchable flexible inner wrap 60; which itself may be encased by a low lint, flexible, fluid permeable outer wrap 62; with the second substrate material 20 being positioned between the inner wrap 60 and the outer wrap 62, wherein the inner wrap 60 and the outer wrap 62 are flexible before insertion and wherein the first material 14 and the stretchable flexible inner wrap 60 encasing the first material 14 are shaped e.g., as a sphere. The inner wrap 60 and the outer wrap 62 material in a pre-assembled state may be a two dimensional rectangular, but other shapes such as trapezoidal, triangular, hemispherical, chevron, hourglass shaped, and circular are acceptable. The inner wrap material and outer wrap material in pre-assembled state may be three dimensional such as cylindrical, cubical, conical, spherical or another multisided shape. The inner wrap material and outer wrap material preferably comprise particles of absorbent foam-like material, e.g., spherical beads having an average diameter of about 0.5 mm to about 6 mm, preferably an average diameter of about 1 mm to about 4 mm.

The outer wrap 62 and inner wrap 60 may measure from about 1 inch to about 5 inches in length and from about 1 inch to about 5 inches in width. The inner wrap 60 is smaller than the outer wrap 62 and can have a measure of from about 0.5 inch to about 4.5 inch in length and from about 0.5 inch to about 4.5 inch in width. Both the inner wrap 60 and outer wrap 62 are preferably flexible. Both the inner wrap 60 and outer wrap 62 may be stretchable or elastic. The inner wrap 60 may be attached or detached from the outer wrap 62. The outer wrap 62 is fluid permeable, while the inner wrap 62 can be fluid permeable or fluid impermeable.

The inner wrap 60 and outer wrap 62 material can be comprised of many materials including woven or non-woven materials or fibers, folded tissues, films such as apertured formed thermoplastic films, apertured plastic films, reticulated thermoplastic films, and hydroformed thermoplastic films or foams such as porous foams and reticulated foams, that may comprise a blend of natural fibers, synthetic fibers or natural and synthetic fibers. The natural fibers include rayon, cotton, wood pulp, flax, and hemp. Acceptable types of rayon include rayon fibers available as 6140 Rayon from Acordis Fibers Ltd., of Hollywall, England (tri-lobed rayon), and SX 275-123 produced by Green Bay Nonwovens, Green Bay, Wis.

The synthetic fibers include fibers such as polyester (e.g., biodegradable polyester) polyolefin, nylon, polypropylene, polyethylene, polyacrylic, polyvinyl acetate, polyacrylate or cellulose acetate. The inner wrap 60 and outer wrap materials can be comprised of Corolind nonwoven material (tradename PE HPC-2, code T23FOR, available from BBA Corp., Simpsonville, S.C., USA).

Another composition of the first and second materials is 50% rayon, 50% polyester hydroentangled (BBA 140027). Alternatively, the inner wrap and outer wrap material can be comprised of a material that is dual layered with an outside and inside layer, e.g., as described in U.S. Pat. No. 5,273,596. In this embodiment, the outside layer is a 75% hydrophilic polypropylene with a 2.2 denier and 25% 1.5 denier rayon. The inside layer is 25% hydrophilic polypropylene with a 2.2 denier and 75% 1.5 denier rayon. The basis weights of the layers can vary. A typical version has from about 10 to about 15 g/m2 in each layer. The resultant material is 50% rayon 50% polypropylene thermally bonded blend with a basis weight from about 20 to about 30 g/m2. These materials are also produced by BBA Corporation.

Fibers in the inner wrap 60 and outer wrap 62 material may have hydrophobic finishes, hydrophilic finishes, or combinations of hydrophobic or hydrophilic finishes. The fibers may be inherently hydrophilic or hydrophobic, or may be treated to provide such properties.

Deformable tampons will preferably have a self-sustaining shape when a tampon pledget has been compressed and/or shaped such that it assumes a general shape and size, which is vaginally insertable, absent external forces. However, a self-sustaining shape preferably does not persist during use of the tampon. Thus, once the tampon is inserted and begins to absorb fluid, the tampon may begin to expand and may lose its most or all of its self-sustaining shape.

Deformable tampons as described above may comprise a first material that is an absorbent material and a second material that is an absorbent material. The absorbent first and second materials can be one or more of cotton, rayon, polysaccharides, comminuted wood pulp (fluff pulp), creped cellulose wadding hydro gel polymer gelling agents, meltblown polymers, carboxymethyl cellulose (optionally cross-linked), polyacrylamide, polyacrylate, peat moss, an absorbent foam, a superabsorbent polymer, an absorbent gelling material, an absorbent sponge and other materials as described in U.S. Pat. No. 7,713,253. In some of these embodiments, the first material 14 will have a first absorbency that is different from a second absorbency the second material 20 has, e.g., the first absorbency is greater than the second absorbency in preferred embodiments. These tampon embodiments may comprise a closer of the outer wrap and a withdrawal member, e.g., a flexible string or fiber material, for removal of the tampon after use. The inner wrap 60 may be fluid permeable.

The sanitary absorbent articles 10 may include malodor control or suppression compounds. Examples of these types of compounds include activated carbons, clays, zeolites, silicates, cyclodextrins, ion exchange resins and mixtures thereof as, for example, described in EP-A-348 978, EP-A-510 619, WO91/12029, WO 91/11977, WO 89/02698, and/or WO 91/12030. These odor control agents are believed to control odor by physically absorbing malodor compounds or their precursors and thus hindering the exit of malodor from the articles. These malodor control or suppression compounds include a pH buffer on top of cationic polysaccharides, preferably chitosan materials, with acidic pH buffer having a pH in the range of from 3.5 to 6.5 as described in U.S. Pat. No. 6,844,430. Such malodor control or suppression compounds can be included with the sanitary absorbent articles 10. The pH levels can be regulated by means of introducing active ingredients such as, for example, carbons, clays, zeolites, silicates, cyclodextrins, ion exchange resins, or any combination thereof. By regulating pH, malodor can be controlled. The configuration of the forementioned manufacturing method, and material properties can create an article that produces little to no lint. Using PVA fibers instead of cotton medical gauze can create high tensile strength and higher strain to failure rates. In addition, biodegradability is very apparent within PVA due to its aerobic and anaerobic conditions. Various applications are done with PVA and have demonstrated high compatibility with minimal to no irritation. (as can be seen in cytotoxicity and skin irritation tests; ISO 10993-5:2010 and ISO 10993-10:2009 respectively).

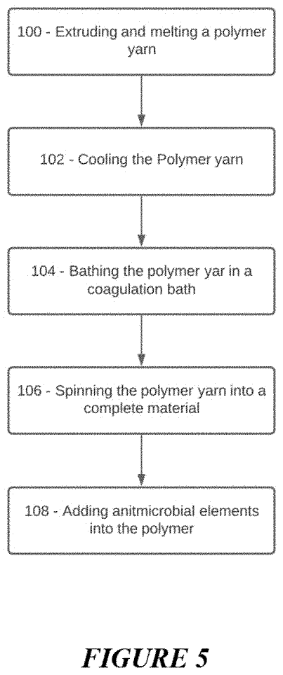

Referring to FIG. 5, a method of manufacturing a sanitary article, the method comprising the acts of extruding a polymer yarn and bathing the extruded polymer yarn in a coagulation bath 100. At 102, spinning the polymer yarn into a textile, at 104 bathing the polymer yarn in a coagulation bath, at step 106 spinning the polymer yarn into a complete material. At step 108 adding antimicrobial elements into the textile. The polymer is polyvinyl acetate. The antimicrobial elements are silver and zinc. The textile is spun into another fabric. The extruded polymer yarn are screw extrusion fibers.

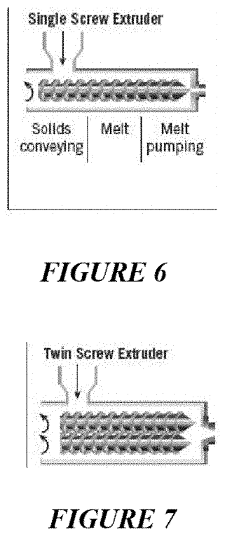

Referring to FIGS. 6 and 7, a sample embodiment of a single extruder and a twin screw extruder used in the manufacturing process of the absorbent sanitary article for body fluid.

The descriptions provided in the claims and other embodiments that are described herein illustrate the invention and they are not intended to limit it to those specific descriptions. All citations herein, e.g., citation to U.S. patents or scientific or other publications, are incorporated herein by reference in their entireties.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

What is claimed is:

1. A system for absorbing a fluid from the body of a user, the system comprising,
an absorbent polymer acting as a substrate for a reducing agent and an oxidizing agent;

2. The system of claim 1 wherein the absorbent polymer comprises a first absorbent polymer acting as a substrate for the reducing agent and a second absorbent polymer acting as a substrate for the oxidizing agent.

3. The system of claim 1 wherein at least one of the oxidizing agent and the reducing agent is coupled to the substrate by a binder wherein the binder degrades in the presence of an electrolytic solution.

4. The system of claim 1 wherein at least one of the reducing agent and the oxidizing agent is positioned on the substrate in a pattern.

5. The system of claim 1 wherein the reducing agent comprises silver and the oxidizing agent includes zinc.

6. The system of claim 1 wherein the ratio of the mass of the reducing agent to the mass of the oxidizing agent is between about 1.7:1 to about 2.3:1

7. The system of claim 3, wherein the ratio of the reducing agent to the binder coupling the reducing agent to the substrate and the ratio of the oxidizing agent to the binder coupling the oxidizing agent to the substrate is between about 2:3 and about 7:3.

8. The system of claim 1 wherein the absorbent polymer is polyvinyl acetate, acrylonitrile polymer, polyacrylic acid, polyacrylamide, polyvinyl alcohol or a hydrogel.

9. The system of claim 1 wherein the absorbent polymer is grafted onto starch.

10. The system of claim 1 further comprising an outer material overlaying the absorbent polymer, wherein at least one of the reducing agent and the oxidizing agent is under the surface of the outer material

11. The system of claim 10 wherein the outer material is selected from the group including an absorbent surface skin, a thin surface, and a surface layer.

12. The system of claim 10 wherein the outer material is an absorbent low lint material.

13. The system of claim 1 wherein the reducing agent comprises silver particles in an amount equal to about 2% to about 6% of the substrate for the reducing agent.

14. The system of claim 1 wherein at least one of the reducing agent and the oxidizing agent resides in a plurality of first reservoirs that are positioned on the substrate.

15. The system of claim 10 wherein the outer material comprises a nonsubstrate absorbent polymer that is not acting as a substrate for reducing agent or oxidizing agent.

16. A method of manufacturing a sanitary article, the method comprising the acts of:
extruding a polymer yarn and bathing the extruded polymer yarn in a coagulation bath;
spinning the polymer yarn into a textile; and
adding antimicrobial elements into the textile.

17. The method of claim 16 wherein the polymer is polyvinyl acetate.

18. The method of claim 16 wherein the antimicrobial elements are silver and zinc.

19. The method of claim 16 wherein the textile is spun into another fabric.

20. The method of claim 15 wherein the extruded polymer yarn are screw extrusion fibers.
